(19) United States
(12) Patent Application Publication
(10) Pub. No.: US 2010/0222734 A1
(43) Pub. Date: Sep. 2, 2010

(54) Double-sided patch

(76) Inventors: Dalia Jayes, Modiin (IL); Gerry Tal, Petach Tikva (IL)

Correspondence Address: KENYON & KENYON LLP, 1500 K STREET N.W., SUITE 700, WASHINGTON, DC, 20005, US

(21) Appl. No.: 11/991,152

(22) PCT Filed: Aug. 28, 2006
(86) PCT No.: PCT/IL06/00991

§ 371 Date: Apr. 11, 2008

Related U.S. Application Data
(60) Provisional application No. 60/712,852, filed on Sep. 1, 2005.
(60) Provisional application No. 60/792,336, filed on Apr. 17, 2006.

(51) Int. Cl. A61N 1/04; A61N 1/30

(52) U.S. Cl. 604/20; 607/152

(57) ABSTRACT

The present invention provides a double-sided patch comprising a first side and a second side, wherein each of the first side and the second side of the double-sided patch is configured as a treatment patch. Furthermore, the present invention provides a double-sided patch, wherein each side of the double-sided patch shares at least one common patch component with the other side of the double-sided patch.

### FIELD AND BACKGROUND OF THE INVENTION

The present invention is related to a double-sided patch, wherein each side of the patch facilitates a treatment, such as generating electricity for electrical stimulation and/or drug delivery.

There is known in the art today a range of dermal patches, which are passive or alternatively include batteries or galvanic coupling means to electrically stimulate a membrane such as skin, teeth, mucosa or hair. Such patches can promote delivery of at least one compound into the membrane facilitating treatment of a variety of medical and cosmetic conditions.

The cost of such patches, which can include many components such as electrodes, conductive fluid, power source, frames and patch body can be expensive. Further, in the case of disposable patches, patch elements such as power sources and electrodes are often not depleted after completion of treatment.

It would be advantageous to have a single device, which facilitates two patch treatments, but which is cheaper than two single patches. Further it would be desirable to have a patch, which can facilitate two different or the same treatments. Still further it would be desirable to have a double-sided patch, wherein each patch side is configured as a treatment patch and wherein both patch sides share at least one common component. The present invention provides such a patch.

### SUMMARY OF THE INVENTION

The present invention provides a double-sided patch comprising a first side and a second side, wherein each of the first side and the second side of the double-sided patch is configured as a treatment patch. Furthermore, the present invention provides a double-sided patch, wherein each side of the double-sided patch shares at least one common patch component with the other side of the double-sided patch.

The present invention also provides a double-sided patch having at least two sides, which may feature a top side and a bottom side, wherein each of the at least two sides of the double-sided patch is configured as a treatment device. In some embodiments each of the two sides is configured as an electricity generating device for electrical stimulation and/or promoting delivery of an active compound. Each side of the double-sided patch may be contacted separately with a body area for treatment of a cosmetic or medical condition. Each side of the double-sided patch may be configured for at least one of the same treatment, a different treatment, electrical stimulation, delivery of at least one substance, delivery of a different at least one substance, delivery of a different dose of at least one substance, active delivery, passive delivery and a combination thereof.

In addition, the present invention provides uses of a double-sided patch for treatment of any suitable cosmetic, dermatological or medical condition. The present invention also provides methods of promoting a double-sided patch of the present invention and promoting the patch for its intended use.

### BRIEF DESCRIPTION OF THE DRAWINGS

FIG. 1 shows a schematic side view of an electricity generating patch of the prior art; FIG. 2 shows a schematic side view of a double-sided patch according to one embodiment of the present invention;

FIG. 3a shows an exploded view of a double-sided patch assembly according to one embodiment of the present invention;

FIG. 3b shows an exploded view of a double-sided patch assembly according to a further embodiment of the present invention.

FIG. 4 shows a double-sided patch with release liner according to one embodiment of the present invention;

FIG. 5 shows a double-sided patch wherein release liner has been removed from one side according to one embodiment of the present invention;

FIG. 6 shows a double-sided patch wherein the first side is attached to the left side of the facial area according to one embodiment of the present invention;

FIG. 7 shows a double-sided patch wherein the protective liner of the second side of the patch is being removed according to one embodiment of the present invention;

FIG. 8 shows a double-sided patch wherein the second side of the patch is attached to the right side of the facial area for a second treatment according to one embodiment of the present invention;

FIG. 9 shows a schematic side view of a double-sided patch wherein the power source is common to both sides of the patch and the electrodes are not shared according to one embodiment of the present invention;

FIG. 10 shows a schematic side view of a double-sided patch wherein the power source and one of the electrodes are shared by both sides of the patch according to one embodiment of the present invention; and

FIG. 11 shows a schematic side view of a double-sided patch wherein the patch is powered by a galvanic couple common to both sides of the double-sided patch according to one embodiment of the present invention.

### DETAILED DESCRIPTION OF THE INVENTION

In one embodiment the double-sided patch of the present invention may include at least one power source for powering both sides of the double-sided patch. In one embodiment, the double-sided patch may include at least one main electrode and at least one counter electrode, wherein the electrodes may be disposed on a base layer substrate frame, and wherein the frame may include a plurality of apertures. The main electrode and counter electrode may be configured to be conductive on both sides of the electrodes and the electrodes may be exposed via the aperture/s in the frame to both sides of the double-sided patch facilitating function as electrodes for both sides of the double-sided patch. In one embodiment, the double-sided patch may include a conductive interface material, such as but not limited to a hydrogel, which may be disposed on each side of the electrodes. In one embodiment, the double-sided patch may include at least one active substance. In one embodiment, the double-sided patch may include a holding means, such as for example a non-woven material or a pad, to which the conductive interface material may be applied and/or to which the at least one active substance may be applied.

In one embodiment, the double-sided patch includes two patch sides, wherein each of the two sides is configured as a separate treatment patch and may share at least one patch component. At least one shared patch component may include, base layer substrate frame, power source, main electrode and counter electrode and combinations thereof.

In one embodiment, each of the two sides of the double-sided patch may share the same base layer substrate frame. In such an embodiment, the other patch components may not be shared, but may be separate/distinct for each side of the double-sided patch.

In one embodiment, each of the two sides of the double-sided patch may share the same base layer substrate frame and at least one electrode. In such an embodiment, the other patch components may not be shared, but may be distinct for each side of the double-sided patch.

In one embodiment, each of the two sides of the double-sided patch may share the same base layer substrate, the same at least one electrode and the same at least one power source.

In one embodiment, a galvanic couple created by the electrode material of the patch may power the double-sided patch, without the need of an additional power source. In such an embodiment of the double-sided patch, both sides of the double-sided patch may share the same electrodes which will provide a galvanic couple to both sides of the double-sided patch.

In one embodiment, one side of the double-sided patch may be configured as a passive patch and the other side of the double-sided patch may be configured as an active patch. In such an embodiment, the passive side of the double-sided patch may not be connected to the power source.

Furthermore, the present invention provides uses of such a double-sided patch for treatment of any suitable cosmetic, dermatological or medical condition, such as but not limited to, for acne treatment, age spots, dermatitis, skin and nail viral, fungal and bacterial infections, onychomycosis, disorders of the hair follicles and sebaceous glands, scaling disease, scars, wounds, cellulite treatment, skin and tooth whitening, pigmentation disorders, sun damaged skin, fine facial lines, laugh lines, aging skin, dry skin, wrinkles, puffy eyes, lifting skin, warts, benign tumors, malignant tumors, pain management, bone healing, facilitating muscle contraction, promoting metabolic processes, increasing blood flow, treating hyperhidrosis, body decoration and as a drug delivery system for delivering any suitable drug or active ingredient to any suitable body region and combinations thereof.

The present invention also provides methods of promoting a double-sided patch of the present invention and promoting the patch for its intended use. As used herein the term ‘promoting’ includes, but is not limited to promoting, instruction, launching, marketing, commercializing and advertising. Examples include, but are not limited to, graphical, visual or audio representations on the product or in shops, magazines, media, newspaper, radio, television, internet, and the like. A method of promoting can include promoting a patch with a first treatment side and a second treatment side. A method of promoting the double-sided patch for its intended use can include promoting the application of each side of the double-sided patch for a separate same or different treatment by each side of the patch.

As used herein the term ‘electricity generating device’ includes, but is not limited to a powered dermal patch, dermal device, patch device, a galvanic stimulation device or any delivery device, or electrical stimulating device, which is powered and can facilitate a change in a skin or body condition, by any suitable mechanism. The term includes, but is not limited to a topical delivery iontophoretic device, topical delivery ultrasonic device, topical delivery RF device, topical delivery micro-needle device, topical physical therapy device, such as, but not limited to TENS, MIMS, electric wound healing device, skin stimulator, neural stimulator, muscle stimulator, oral care stimulator diffuse surface treatment device and a combination thereof.

As used herein the term ‘treatment’ includes, but is not limited to prevention, elimination, reducing severity, alleviating direct symptoms and related symptoms and curing of a condition. The term also includes pretreatment and post treatment.

As used herein the term ‘at least one substance’ includes but is not limited to an active substance, including any ‘active formulation’, ‘active composition’, ‘active agent’, pharmaceutical, drug, cosmeceutical, cosmetic substance, therapeutic substance, decorative ink, natural and synthetic, which has an effect on any condition, such as, but not limited to a physical, physiological, biochemical, biological, chemical condition or a combination thereof This includes a therapeutic effect, cosmetic effect, an inhibitory effect, stimulatory effect, physical effect, biological effect, physiological effect, preventative effect, placebo effect or combination thereof. In addition, the term may include an inactive substance.

The present invention provides a double-sided patch wherein at least one of the patch components are shared by (common to) each patch side and wherein the double-sided patch, can be configured for a plurality of treatments. Production of a double-sided patch of the present invention may be less costly than producing two separate patches.

The principles and operation of a double-sided patch according to the present invention may be better understood with reference to the figures. The figures show non-limiting embodiments of the present invention.

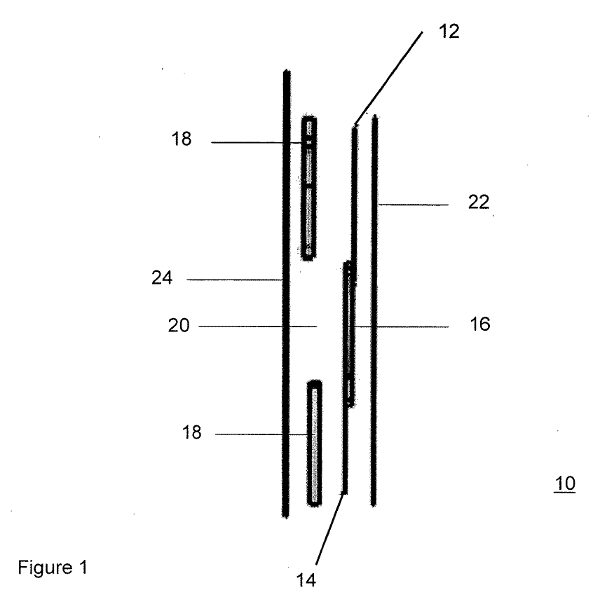

FIG. 1 shows a schematic side view of a patch 10 as described in the prior art. As can be seen from FIG. 1, patch 10 includes a positive electrode 12, a negative electrode 14, a power source 16, conductive material/fluid 18, a patch body 20, and a patch frame 22. Positive electrode 12 and negative electrode 14 are electrically coupled to the power source 16 and conductive fluid 18 is disposed on one side of the electrodes 12, 14. The components are disposed on a patch body 20 in spaced relation to each other. The conductive fluid 18 can include at least one active substance, which can be dermally, intradermally or transdermally delivered for treatment of a cosmetic or medical condition.

In some embodiments, the patch 10 may include a liner 24, which may be removed before the patch 10 may be applied to the body area to be treated. The patch 10 is configured to electrically stimulate the contacted body area and in an embodiment wherein the patch 10 includes at least one substance, the patch may promote delivery of the at least one substance. After treatment is terminated in an embodiment wherein the patch 10 is not reusable, the patch may be discarded.

FIG. 2 shows a schematic side view of a two sided (double-sided) treatment patch 100 according to an embodiment of the present invention. As can be seen from FIG. 2, patch (double-sided patch) 100 includes, at least one positive electrode 102, at least one negative electrode 104, at least one power source 106, a separating base substrate layer frame 108 including at least one aperture 110, a plurality of conductive material formulations 112, a plurality of release liners 114 and two patch body sides 116, 118. In the embodiment shown in FIG. 2, the same at least one power source 106 is used by both sides 116, 118 of the double-sided patch, the same at least one negative electrode 104 and at least one positive electrode 102 are used/shared by both sides 116, 118 of the double-sided patch 100 and the same one base substrate layer frame is used by both sides 116, 118 of the double-sided patch 100. In one embodiment, the at least one negative electrode 104 and the at least one positive electrode 102 are conductive on both sides of the electrode. Electrodes 102, 104 may be disposed on the base layer substrate, wherein the base layer substrate 108 may include at least one aperture 110 or other suitable means for facilitating exposure of the electrodes 102, 104 to both sides 116, 118 of the patch 100. Optionally, base layer substrate 108 may include any other suitable means in combination with the at least one aperture 110 or instead of at least one aperture 110 to facilitate conduction of the electrodes 102, 104 to both sides 116, 118 of the patch 100. Examples of suitable means include use of a base layer substrate material 108, which is conductive in the areas in contact with the electrodes, such as by use of a conductive via or a suitable conductive material. In the embodiment shown in FIG. 2, electrodes 102, 104 are configured to function on both sides 116, 118 of the patch 100, facilitating the same electrodes 102, 104 to conductively couple with a body area on both sides 116, 118 of the double-sided patch. In such a way the conductive interfacing means 112, such as a conductive fluid 112, for example a hydrogel can be disposed on both sides of the positive electrode 112(i), 112(ii) and the negative electrode 112(iii), 112(iv) and on both sides 116, 118 of the patch 100. Conductive material 112 can include at least one active substance for one or a combination of dermal, intradermal or transdermal delivery and delivery to the stratum corneum and epidermis for treatment of a cosmetic or medical condition or facilitating a cosmetic effect. In some embodiments, double-sided patch may include at least one holding means (not shown in figure), such as for example a non-woven material or pad for accommodating the conductive material 112 and/or at least one active substance. In some embodiments, only one side of the double sided patch may include the at least one holding means. In some embodiments, conductive interfacing means 112 and/or at least one active substance may not be integrally formed with the patch, but may be part of a kit and may be applied in any suitable way to any suitable patch component or body area before treatment.

In an embodiment, wherein both sides 116, 118 of the patch 100 include different at least one active ingredients, one side of the patch 116 can be for a first type of treatment of a body area condition and the second side of the patch 118 can be for a second type of treatment of a body area. One non-limiting example is wherein one side of the patch 100 includes a local anaesthetic for desensitizing a body area and the second side of the patch 100 includes an active substance, such as a skin whitening agent. A second non limiting example is wherein one side 116 of the patch does not include an active agent and is configured for electrical stimulation of a body area. A second side of the patch 118 can include an active agent, which can be delivered to the body area to facilitate treatment. A third non-limiting example is wherein one side of the patch 116 is an active patch and is configured to treat the body area by electrical stimulation and/or delivery of an active substance and the second side 118 of the double-sided patch is configured as a passive patch. The passive patch side may facilitate transdermal delivery of an active substance.

One side of the double-sided patch 100 can be applied to a desired body area and treatment initiated. Treatment with the first one side of the double-sided patch may be terminated, such as by removing the double-sided patch from the body area. In some embodiments, the first side, after being used, may be covered by a covering means, such as, but not limited to, by a liner, which may be the original liner, a second side of the original liner, or a different liner, which may be marked for this function. Optionally, instead of, or in addition to, applying a covering means to the first used side, the conductive interfacing means 112 may be removeable and can be removed from the used side of the double-sided patch. A covering means or removal of the conductive interfacing means 112 of the used side of the double-sided patch facilitate hygienic and ease of use of the second side of the double-sided patch, reducing or eliminating contact by a user with the conductive interfacing means 112 of the used side when applying the second side of the double-sided patch. The second side of the double-sided patch, which has not been applied to a body area can be applied to the same body area as was treated by the first side of the double-sided patch or to a different body area. The second side of the double-sided patch may be applied immediately after treatment with the first side of the double-sided patch or at any convenient later time. As described above both sides of the double-sided patch can be configured for the same treatment or for different treatments, for the same body area or for different body areas.

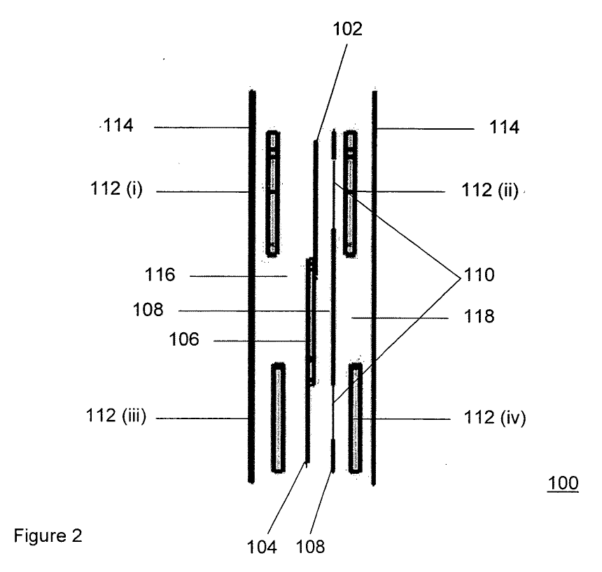

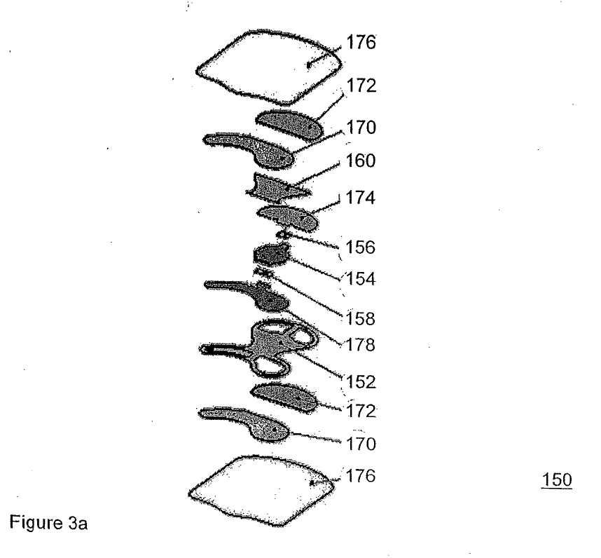

FIG. 3a shows an exploded view of a double-sided patch assembly according to one embodiment of the present invention. As can be seen in FIG. 3a, double-sided patch device 150 includes a release liner 176 disposed on a conductive material 172 and a conductive material 170. In some embodiments the conductive materials 170, 172 are hydrogels. The conductive material 172 is disposed on a counter electrode 174 and the conductive material 170 is disposed on a main electrode 178. The patch 150 may further include a battery cover 160. The counter electrode 174 may be connected by a conductive adhesive strip 156 to a battery 154, and wherein the battery 154 is also connected by a conductive adhesive strip 158, to the main electrode 178. In the embodiment shown in FIG. 3a, the double-sided patch 150 includes a base layer substrate frame with a plurality of openings 152. The base layer substrate frame with a plurality of openings 152 is disposed on the main electrode 178 and the counter electrode 174, such that the electrodes 174, 178 are exposed through the aperture to a second side of the frame 152 and the double-sided patch 150. In the embodiment shown in FIG. 3a, which is non-limiting, the base layer substrate with a plurality of openings 152 includes four openings (apertures). The main electrode 178 is disposed over two of the frame openings and the counter electrode 174 is disposed over the other two frame openings. On the second side of the frame 152, the conductive material 172 such as hydrogel is disposed on the second side of the counter electrode 174, and the conductive material 170 such as hydrogel is disposed on the second side of the main electrode 178. A second release liner 176 may be disposed on hydrogel 170, 172.

FIG. 3b shows an exploded view of a double-sided patch assembly 200 according to one embodiment of the present invention. From FIG. 3b it can be seen that a power source 208 includes a negative tab 210 and a positive tab 206. The negative tab 210 may be coupled to a counter electrode 212 and the positive tab 206 may be coupled to a main electrode 204. As such both the counter electrode 212 and the main electrode 204 may be coupled to a power source 208. In one embodiment, the counter electrode 212 and the main electrode 204 are conductive on both sides. The double-sided patch 200 may include a frame 202. The frame 202 may include a plurality of apertures or other suitable means, such that the electrodes 212 and 204 may be configured for use on both sides of the frame 202. The frame 202 may facilitate exposure of regions of the electrodes 212 and 204 to both sides of the frame 202. In the non-limiting embodiment shown in FIG. 3b, the frame 202 includes 2 apertures. Each side of the frame 202 may be configured as a different side of the double-sided patch 200 and may facilitate a separate treatment. In some embodiments, the frame 202 may be constructed from a non-conductive material. In some embodiments regions of the frame in contact with the electrodes 204 and 212 may be conductive in order to facilitate use of the electrodes 204 and 212 on both sides of the frame 202. A conductive material 214, 216, such as hydrogel may be disposed on both sides of the electrodes 204 and 212. A main hydrogel 214 may be disposed on the main electrode 204 and a counter hydrogel 216 may be disposed on the counter electrode 212. The hydrogel 214, 216 may include at least one active ingredient such as a drug or cosmetic or any suitable formulation and excipient. The hydrogel 214, 216 disposed on the different sides of the frame 202 may include the same or different active ingredients in the same or different doses. A release liner may be disposed on exposed hydrogel regions 214 and 216. One side of the double-sided patch/frame may include a release liner 220 and the other side of the double-sided patch may include a release liner 218. The release liners 218 and 220 may be made of the same or different materials. Double sided patch may include a battery cover 222.

In certain embodiments, Item 202 can be made of 3M 9907W.

In certain embodiments, Item 204 can be made of Intelicoat 7101.

In certain embodiments, Item 206 can be made of AR 90447.

In certain embodiments, Item 210 can be made of AR 90447.

In certain embodiments, Item 212 can be made of Intelicoat 8101.

In certain embodiments, Item 214 can contain FW 200.

In certain embodiments, Item 216 can contain FW 200.

In certain embodiments, Item 218 can be made of HDPE.

In certain embodiments, Item 220 can be made of DuPont Purex U32.

In certain embodiments, Item 222 can be made of 3M 9907W.

The double-sided device of the present invention may be used to deliver almost any active substance/drug and formulation thereof. Non-limiting examples include therapeutic substances in all of the major therapeutic areas including, but not limited to, antiinfectives such as antibiotics and antiviral agents, analgesics including fentanyl, sufentanil, buprenorphine and analgesic combinations, anesthetics, anorexics, antiarthritics, antiasthmatic agents such as terbutaline, anticonvulsants, antidepressants, antidiabetic agents, antidiarrheals, antihistamines, antiinflammatory agents, antimigraine preparations, antimotion sickness preparations such as scopolamine and ondansetron, antinauseants, antineoplastics, antiparkinsonism drugs, cardiostimulants such as dobutamine, antipruritics, antipsychotics, antipyretics, antispasmodics; including gastrointestinal and urinary, anticholinergics, sympathornimetics, xanthine derivatives, cardiovascular preparations including calcium channel blockers such as nifedipine, beta-blockers, beta-agonists such as salbutamol and ritodrine, antiarrythmics, antihypertensives such as atenolol, ACE inhibitors, diuretics, vasodilators, including general, coronary, peripheral and cerebral, central nervous system stimulants, cough and cold preparations, decongestants, diagnostics, hormones such as parathyroid hormone, growth hormone and insulin, hypnotics, immunosuppressives, muscle relaxants, parasympatholytics, parasympathomimetics, anti-oxidants; nicotine, prostaglandins, psychostimulants, sedatives and tranquilizers, herbal preparations and homeopathic remedies.

The double-sided device of the present invention is particularly useful for the delivery of cosmetic and cosmeceutical substances. Such substances, include, for example, skin acting anti-oxidants, such as caretenoids, ascorbic acid (vitamin C) and vitamin E, as well as other vitamin preparations and other anti-oxidants; anti wrinkling agents such as retinoids, including retinol (vitamin A alcohol), alpha-hydroxic acids, beta-hydroxy acid, better known as salicylic acid, skin peeling agents, combination-hydroxy acids and poly-hydroxy acids, and hydrolyzed and soluble collagen and others; moisturizers such as hyaluronic acid, water and others; anticellulite agents and acetyl-hexapeptide-3 (Argireline), pentatpeptide-3, palmitoyl-tetrapeptide-3, GHK, Myoxinol LS, N6 and Boswellic acids, caffeine and skin whitening agents such as arbutin.

Active substances for the treatment of skin disorders of dermatological nature may be selected from the group comprising antibiotic, antibacterial, antifungal, antiviral, anesthetic, analgesic, antiallergic, corticosteroid, retinoid, anti-histamine, sulfur, immunosuppressant and antiproliferative medications, and mixtures thereof at any proportion.

It is understood that the double-sided device of the present invention may be used for delivery of a wide range of dosages of the above listed and other substances over a desired duration of time. The concentration of the active substances may be adopted to exert an effect including a therapeutic effect on a condition when applied to an afflicted area.

Examples of skin disorders of cosmetic nature for which the double-sided device of the present invention may be used include the following: aging skin, facial lines, laugh lines, dry skin, sun damaged skin, wrinkles, age spots, various hyperpigmented spots, melasma, puffy eyes, scars, warts, varicose veins, stretch marks, under eye dark circles, lifting skin, lip plumping, acne, redness of the skin, telangiectasia, hair growth disorders, cellulite, and obesity and combinations thereof.

The double-sided device of the present invention may also find use in increasing blood flow, pain management, onychomycosis, wound treatment, bone healing, treating hyperhidrosis, facilitating muscle contraction, for massage and promoting metabolic processes. Examples of skin disorders of dermatological nature, as well as active substances which may be used to treat them, are set forth in Table 1.

TABLE 1
A non-exhaustive listing of dermatological disorders,
suitable for usage of the double-sided device of the
present invention and exemplary drugs for such disorders.

| | Exemplary Active |
|---|---|
| Dermatological Disorder | Substance |
| Dermatitis | Steroidal and non-steroidal |
| Contact Dermatitis | anti-inflammatory agents |
| Atopic Dermatitis | |
| Seborrheic Dermatitis | |
| Nummular Dermatitis | |
| Chronic Dermatitis Of The | |
| Hands And Feet | |
| Generalized Exfoliative | |
| Dermatitis | |
| Stasis Dermatitis | |
| Bacterial Infections Of The Skin | Antibiotic and anti- |
| Cellulitis | inflammatory agents |
| Acute Lymphangitis | |
| Lymphadenitis | |
| Erysipelas | |
| Cutaneous Abscesses | |
| Necrotizing Subcutaneous Infections | |
| Staphylococcal Scalded Skin | |
| Syndrome | |
| Folliculitis | |
| Furuncles | |
| Hidradenitis Suppurativa | |
| Carbuncles | |
| Paronychial Infections | |
| Erythrasma | |
| Fungal Skin Infection | Antifungal agents |
| Infections caused by dermatophytes--fungi | |
| that invade only dead tissues of the skin or | |
| its appendages (stratum corneum, nails, hair) | |
| onychomycosis | |
| Infections of skin (usually of moist, | |
| occluded, intertriginous areas), skin | |
| appendages, or mucous membranes caused | |
| by yeasts of the genus Candida. | |
| Viral Skin Infection | Antiviral agents |
| Warts | |
| Herpes | |
| Disorders of the Hair Follicles And | Keratolytic agents |
| Sebaceous Glands | |
| Acne | antibiotics |
| Rosacea | Anti-inflammatory agents |
| Perioral Dermatitis | Sulfur |
| Hypertrichosis | |
| Alopecia | |
| Pseudofolliculitis Barbae | |
| Keratinous Cyst | |
| Scaling Papular Diseases | Steroidal and non-steroidal |
| Psoriasis | anti-inflammatory agents |
| Pityriasis Rosea | Anti-proliferative agents |
| Lichen Planus | |
| Pityriasis Rubra Pilaris | |
| Pigmentation Disorders | Melanin synthesis |
| Hypopigmentation | inhibitors and enhancers |
| Hyperpigmentation | |
| Scars | Retinoids (e.g., |
| | retinoic acid) |
| | Alpha and |
| | beta hydroxy |
| | acids |
| Warts | Keratolytic |
| | agents |
| Benign Tumors | Keratolytic |
| Moles | agents |
| Dysplastic Nevi | Antibiotics |
| Skin Tags | Anti- |
| Lipomas | inflammatory |
| Angiomas | agents |
| Pyogenic Granuloma | |
| Seborrheic Keratoses | |
| Dermatofibroma | |
| Keratoacanthoma | |
| Keloid | |
| Malignant Tumors | Various anticancer agents |
| Actinic keratosis (pre-cancer | Photodynamic therapy |
| condition) | agents and precursors (e.g., |
| Basal Cell Carcinoma | porphirins and ALA) |
| Squamous Cell Carcinoma | Nonsteroidal anti |
| Malignant Melanoma | inflammatory drugs |
| Paget's Disease Of The Nipples | (NSAID) |
| Kaposi's Sarcoma | |

Treatment according to the present inventions may be beneficial in all body areas. The double-sided device may be of any suitable size and thickness. The double-sided device may include components, which may be made thin and flexible, such as a thin and flexible power source and/or thin and flexible electrodes. Any suitable thin and flexible power source may be used. In some embodiments, thin and flexible power source, comprises batteries produced by Power Paper Ltd. (Petah-Tikva, Israel). Such thin and flexible batteries are described, for example, in U.S. Pat. Nos. 5,652,043, 5,897,522 and 5,811,204, whose disclosures are incorporated herein by reference. Such batteries may include an open electrochemical cell comprising a first layer of insoluble negative pole, a second layer of insoluble positive pole and a third layer of wet electrolyte, the third layer being disposed between the first and second layers and including a deliquescent material, an electroactive soluble material for obtaining required ionic conductivity and a polymer adhesive agent. Additional details can also be found at www.powerpaper.com. Thin batteries of this sort are typically less than 1 mm thick.

The double-sided patch of the present invention may be made by any suitable method, including using a suitable printing technique or a pick and place method. In one embodiment of the present invention, a frame may be constructed from any suitable material, such as a non-conductive material. One non-limiting example of a suitable non-conductive material is polyester. The frame may be constructed with a plurality of apertures, such as for example two apertures, which are configured as the means to facilitate electrode conduction on both sides of the double-sided patch. The apertures may be made by cutting of the frame material. The frame may include an adhesive material to facilitate adhering components to it.

A counter electrode may be applied to the frame, such that it covers totally or partially one of the apertures in the frame. The counter electrode may be any suitable counter electrode made using any method of the art, which may be cut into the desired shape and may be applied in any suitable way to the frame. One example of a suitable counter electrode is a silver/silver chloride electrode, which may be applied using an adhesive or a printing method.

A power source may be applied to the frame. Any suitable power source may be used. In some embodiments the power source is a thin and flexible power source as described herein. The power source may be cut into the desired shape and attached to the frame using any suitable attaching means. In one embodiment, the power source is printed directly onto the frame material.

A main electrode may be applied to the frame, such that it covers totally or partially at least one aperture in the frame, which is not covered by the counter electrode. The main electrode may be any suitable main electrode made using any method of the art, which may be cut into the desired shape and may be applied in any suitable way to the frame. One example of a suitable main electrode is a graphite electrode, which may be applied using an adhesive or a printing method.

The battery may be connected to the counter electrode and the main electrode using any suitable connection means. One non-limiting example of suitable connection means is a conductive adhesive applied onto the frame, such that it couples the battery terminals to the respective electrode.

A conductive interfacing material may be applied to each side of the electrodes. One non-limiting example of a conductive interfacing material is a hydrogel, which may be cut into a desired shape, such as in the shape of the electrodes and which may be applied by any suitable method, to both sides of the counter electrode and main electrode.

Release liners may be constructed from any suitable material such as, but not limited to polyester coated with silicone and may be applied to either side of the double-sided patch in order to protect the treatment sides before use.

The double-sided device may be made by a printing technique, wherein the power source, the electrodes, the connections and the conductive formulation are printed in any suitable way using any suitable printing technique. In an embodiment wherein the double-sided devices are thin, flexible and versatile in shape and form, the double-sided devices of the present invention can be designed to fit any area of the body and to have any desirable size, according to the affected area.

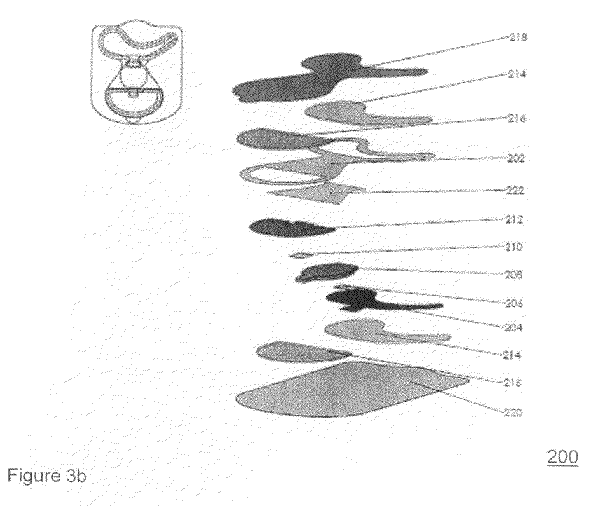

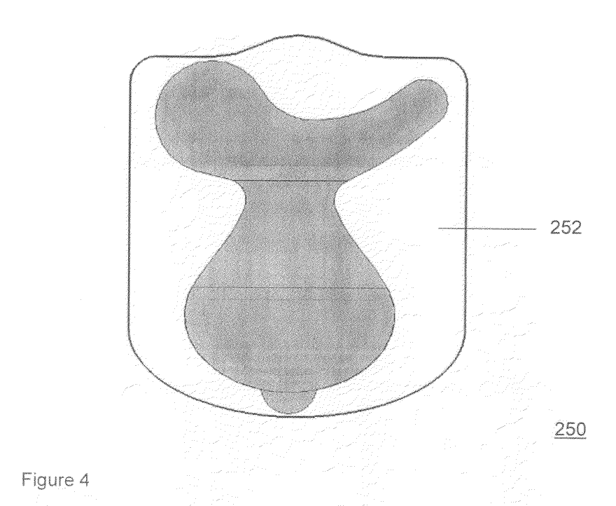

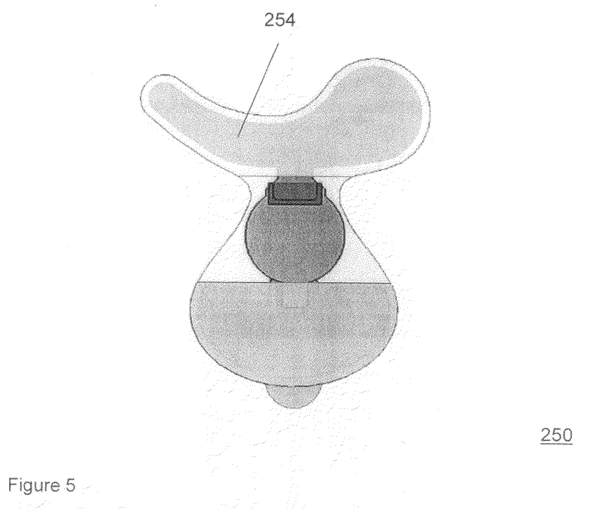

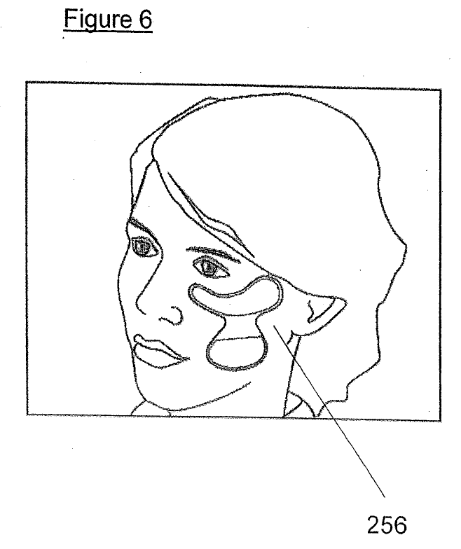

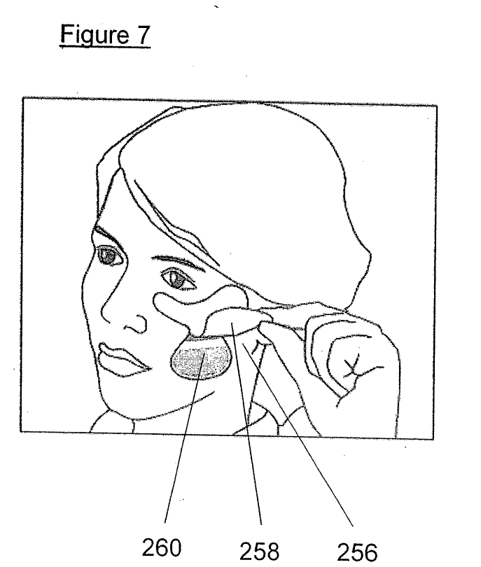

FIG. 4 shows a double-sided patch 250 with an attached release liner 252 according to one embodiment of the present invention. In one non-limiting example, the release liner 252 may be released by a user, to facilitate a double-sided patch with one patch side 254 without a release liner and with an exposed conductive interfacing material, such as a hydrogel as shown in FIG. 5. The first side of the double-sided patch without the release liner 254 and with the exposed hydrogel may be attached to any suitable body area, such as a first side of the face 256, as is shown in FIG. 6. Attachment to the first side of the face 256 facilitates treatment of the first side of the face. FIG. 6 shows the first patch side attached to the left side of the facial area 256. After treatment is completed on the first side of the face or at any suitable time, a protective liner 258 on a second unused side 260 of the double-sided patch may be removed, such as shown in FIG. 7 and the second patch side 260, which may be configured for use on another body area such as the right hand side of the face may be attached to the other body area such as the right hand side of the facial area 262 as shown in FIG. 8. The second patch side 260 may facilitate a second treatment.

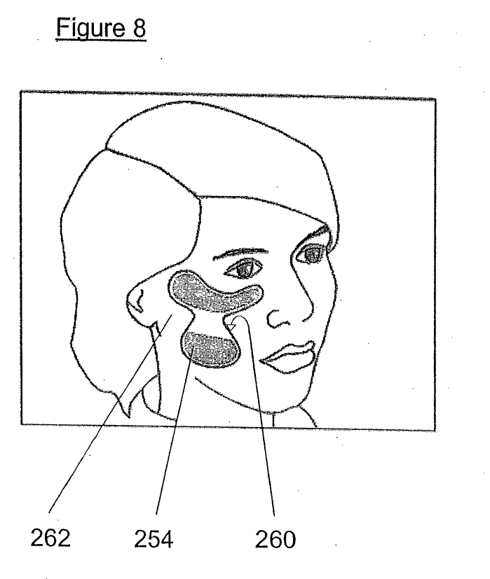

FIG. 9 shows a schematic side view of a double-sided patch wherein the power source is common to both sides of the patch and the electrodes are not shared according to one embodiment of the present invention. As can be seen from FIG. 9, the double-sided patch 300 may include, at least one common power source 302 shared by both sides of the double-sided patch 300, a first patch side anode 304, a first patch side cathode 306, a plurality of conductive formulations 308 disposed on the electrodes of the first patch side, a second patch side anode 310, a second patch side cathode 312, a plurality of conductive formulations 314 disposed on electrodes of the second side of the patch, a separating base substrate layer frame 316 including a means whereby a power source is coupled to both sides of the double-sided patch, a release liner 318 on the first side of the patch and a release liner 320 on the second side of the patch. In an alternative embodiment, the conductive formulation 308, 314 may not be integrally formed with the double-sided patch, but may be part of a kit and may be applied in any suitable way to any suitable patch component or body area before treatment. In some embodiments the conductive formulation 308, 314 may include an active ingredient as detailed hereinabove.

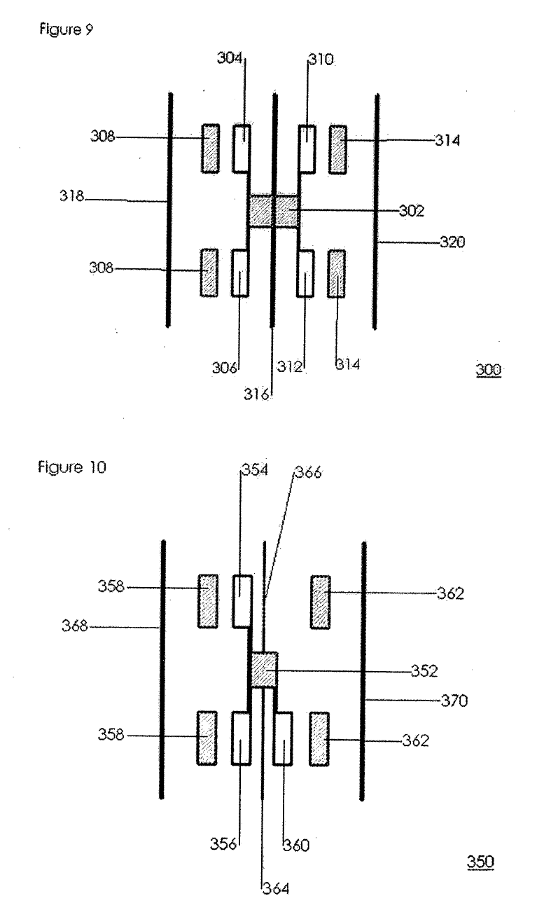

FIG. 10 shows a schematic side view of a double-sided patch wherein the power source and one of the electrodes are shared by both sides of the double-sided patch according to one embodiment of the present invention. As can be seen from FIG. 10, the double-sided patch 350 may include, at least one common power source 352 shared by both sides of the double-sided patch 350, one electrode 354 shared by both sides of the double-sided patch, a first patch side electrode 356 of opposite polarity than the shared electrode, a plurality of conductive formulations 358 disposed on the electrodes of the first patch side, a second patch side electrode 360 of opposite polarity than the shared electrode, a plurality of conductive formulations 362 disposed on the electrodes of the second side of the patch, a separating base substrate layer frame 364 including a means whereby the power source is coupled to both sides of the double-sided patch and including a means 366, such as detailed herein above to facilitate conduction of the shared electrode 354 to both sides of the double-sided patch, a release liner 368 on the first side of the double-sided patch and a release liner 370 on the second side of the double-sided patch. The electrode 354 shared by both sides of the double-sided patch may be either the anode or the cathode, the main electrode or the counter electrode.

In an alternative embodiment, the conductive formulation 358, 362 may not be integrally formed with the double-sided patch, but may be part of a kit and may be applied in any suitable way to any suitable patch component or body area before treatment. In some embodiments the conductive formulation 358, 362 may include an active ingredient as detailed hereinabove.

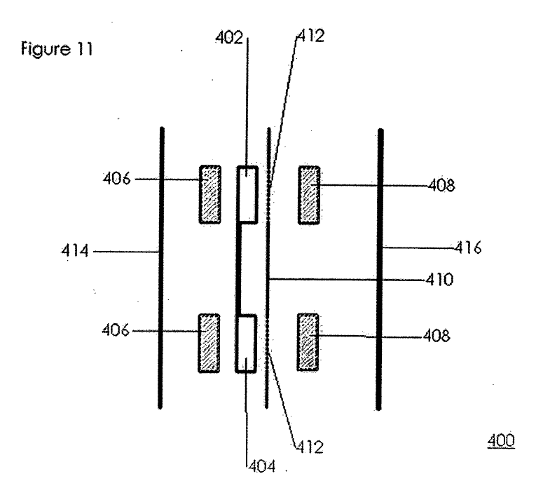

FIG. 11 shows a schematic side view of a double-sided patch wherein the double-sided patch is powered by a galvanic couple common to both sides of the double-sided patch according to one embodiment of the present invention. As can be seen from FIG. 11, double-sided patch 400 may include, at least one anode 402 shared by both sides of the double-sided patch 400, at least one cathode 404 shared by both sides of the double-sided patch, wherein the cathode 404 and the anode 402 are chosen such that the difference in potential of the materials of the electrodes 402 and 404 facilitate a galvanic couple to power the double-sided patch. The double-sided patch further includes a plurality of conductive formulations 406 disposed on the first patch side, a plurality of conductive formulations 408 disposed on the second side of the patch, a separating base substrate layer frame 410 including at least one means 412, such as detailed herein above to facilitate conduction of the shared electrodes to both sides of the patch. The double-sided patch may also include a release liner 414 on the first side of the double-sided patch and a release liner 416 on the second side of the double-sided patch. In an alternative embodiment, the conductive formulation 406, 408 may not be integrally formed with the double-sided patch, but may be part of a kit and may be applied in any suitable way to any suitable patch component or body area before treatment. In some embodiments the conductive formulation 406, 408 may include an active ingredient as detailed hereinabove.

Reference is now made to the following example, which together with the above descriptions illustrate the invention in a non-limiting fashion.

### Example

Production of a Double-Sided Patch

A frame was constructed by cutting the frame material, such as double-sided adhesive polyester into a frame shape including two apertures. A silver/silver chloride counter electrode was applied onto both sides of an electrode substrate using a printing technique. The silver/silver chloride electrode was then cut into a suitable shape and placed on the frame, such that the silver/silver chloride electrode covered one of the apertures in the frame. The counter electrode was attached to the frame by the adhesive of the frame. A negative tab made from conductive adhesive material was cut into a suitable shape and assembled onto the silver/silver chloride electrode. A thin and flexible battery manufactured by Power Paper (STD8/8P) was cut into a suitable shape and placed on the frame such that the negative tab was in contact with a negative terminal of the battery. A positive tab made from conductive adhesive was cut into a suitable shape and applied such that it was in contact with a positive terminal of the battery. A main graphite electrode was applied onto both sides of an electrode substrate using a printing technique. The graphite electrode was then cut into a suitable shape and applied using an adhesive onto the frame, such that the graphite electrode covered the second aperture in the frame and such that the graphite electrode was in contact with the positive tab. A piece of hydrogel was cut into a shape suitable for application onto the graphite electrode and applied onto one side of the graphite electrode. An additional piece of hydrogel was cut into a shape suitable for application onto the silver/silver chloride electrode and placed on one side of the silver/silver chloride electrode. A second piece of hydrogel was cut into a shape suitable for application onto the graphite electrode and was placed on the second side of the aperture in contact with the graphite electrode. A second piece of hydrogel was cut into a shape suitable for application onto the silver/silver chloride electrode and was placed on the second side of the aperture in contact with the silver/silver chloride electrode. Two release liners were cut into a suitable shape and each placed over either side of the assembled double-sided patch.

It is to be understood that the invention is not limited in its application to the details of construction and the arrangement of the components set forth in the description. The invention includes other embodiments and can be practiced or implemented in various ways. Also it is to be understood that the phraseology and terminology employed herein is for the purpose of description only and should not be regarded as limiting.

1. A double-sided patch comprising a first top side and a second bottom side, wherein each of the top side and the bottom side of the double-sided patch is configured as a separate treatment patch.

2. The double-sided patch of claim 1, further comprising at least one main electrode and at least one counter electrode and wherein both sides of the main electrode and the counter electrode are conductive and wherein at least one of the same at least one main electrode and at least one counter electrode are configured as electrodes for both the top and the bottom sides of the double-sided patch.

3. The double-sided patch of claim 2, comprising a power source electrically coupled to the at least one main electrode and the at least one counter electrode.

4. The double-sided patch of claim 3, wherein both the top and the bottom sides of the double-sided patch are powered by the same power source.

5. The double-sided patch of claim 2, wherein the at least one main electrode and the at least one counter electrode provide a galvanic couple.

6. The double-sided patch of claim 5, wherein both sides of the double-sided patch are powered by the current flow resulting from the galvanic couple.

7. The double-sided patch of claim 2, further comprising a base substrate frame, wherein the base substrate frame comprises at least one means configured to facilitate conductive coupling of the at least one main electrode and the at least one counter electrode to both the top and the bottom sides of the double-sided patch.

8. The double-sided patch of claim 7, wherein the at least one means comprises at least one aperture.

9. The double-sided patch of claim 8, wherein the at least one main electrode and the at least one counter electrode are disposed on the base substrate frame and wherein at least part of the at least one main electrode and the at least one counter electrode are disposed on the at least one aperture of the frame facilitating electrode function on both the top and the bottom sides of the double-sided patch.

10. The double-sided patch of claim 2, further comprising at least one conductive material.

11. The double-sided patch of claim 10, wherein the at least one conductive material includes at least one active substance.

12. The double-sided patch of claim 10, wherein the at least one conductive material comprises a hydrogel.

13. The double-sided patch of claim 10, wherein the at least one conductive material is disposed on each side of the at least one main electrode and/or the at least one counter electrode.

14. The double-sided patch of claim 13, wherein each of the conductive material disposed on the two sides of the at least one main electrode comprises at least one active substance.

15. The double-sided patch of claim 14, wherein the at least one active substances in the conductive materials disposed on the two sides of the at least one main electrode are selected from the group consisting of (a) the same active substance with the same dose, (b) the same active substance with different doses, (c) different active substances with the same dose, (d) different active substances with different doses, and (e) a combination thereof.

16. The double-sided patch of claim 13, further comprising a release liner disposed on each of the top side and bottom side of the double-sided patch on the at least one conductive material.

17. The double-sided patch of claim 1 wherein the first top side treatment patch and the second bottom side treatment patch include at least one common shared component.

18. The double-sided patch of claim 17, wherein the at least one common shared component comprises at least one or a combination of at least one counter electrode, at least one main electrode, a base layer substrate frame, at least one conductive material, and at least one power source.

19. The double-sided patch of claim 17, wherein the at least one common shared component comprises a base layer substrate frame.

20. The double-sided patch of claim 17, wherein the at least one common shared component comprises a base layer substrate frame and at least one power source.

21. The double-sided patch of claim 17, wherein the at least one common shared component comprises a base layer substrate and at least one counter electrode and at least one main electrode.

22. The double-sided patch of claim 17, wherein the at least one common shared component comprises a base layer substrate, at least one counter electrode, at least one main electrode and at least one power source.

23. The double-sided patch of claim 1, wherein at least one of the first top side treatment patch and the second bottom side treatment patch comprises an active patch.

24. The double-sided patch of claim 1, wherein at least one of the top and bottom treatment patch sides comprises a passive patch.

25. The double-sided patch of claim 11, further comprising a holding means to accommodate the at least one conductive material and/or at least one active substance.

26. A double-sided patch with a top side and a bottom side comprising:
a main electrode, wherein the main electrode is conductive on both sides of the electrode;
a counter electrode, wherein the counter electrode is conductive on both sides of the electrode and wherein the main electrode and counter electrode are disposed in spaced relation to each other;
a power source electrically coupled to the main electrode and the counter electrode; and
a base layer substrate frame comprising at least one means configured to facilitate coupling of the main electrode and the counter electrode to both the top side and the bottom side of the double-sided patch on which the main electrode and the counter electrode are disposed and whereby the main electrode and the counter electrode are exposed to both the top and bottom sides of the double-sided patch.

27. The double-sided patch of claim 26 further comprising at least one conductive material disposed on both sides of the main electrode and the counter electrode.

28. The double-sided patch of claim 27, wherein the conductive material comprises at least one active substance.

29. The double-sided patch of claim 26, wherein the at least one means configured to facilitate coupling of the main electrode and the counter electrode to both sides of the double-sided patch comprises at least one aperture.

30. A kit comprising a double-sided patch of claim 1 and a conductive formulation.

31. A method of using a double-sided treatment patch comprising:
applying a first top side of a double-sided patch to a body area, wherein the double-sided patch comprises: a top side and a bottom side and wherein each of the top side and the bottom side of the double-sided patch is configured as a separate treatment patch;
removing the first side of the double-sided treatment patch from the body area at the end of a treatment time;
applying a second bottom side of the double-sided patch to a body area; and
removing the second side of the double-sided treatment patch from the body area at the end of a treatment time.

32. The method of claim 31, further comprising removing a release liner before use of the first and the second sides of the double-sided patch.

33. A method of promoting a double-sided patch comprising a first top side and a second bottom side, wherein each of the first side and the second side of the double-sided patch is configured as a separate treatment patch, the method comprising promoting a double-sided patch with a first top treatment side and a second bottom treatment side.

34. The method of claim 33, the method comprising promoting the application of each side of the double-sided patch for a separate treatment by each side of the double-sided patch.

35. A method of making a double-sided patch comprising:
providing a main electrode;
providing a counter electrode;
providing a frame material comprising at least one means configured to facilitate coupling of the main electrode and the counter electrode to both the top and bottom sides of the double-sided patch;
applying the main electrode to the frame and disposing the main electrode on the at least one means configured to facilitate coupling of the main electrode to both the top and bottom sides of the double-sided patch;
applying the counter electrode to the frame and disposing the counter electrode on the at least one means configured to facilitate coupling of the counter electrode to both the top and bottom sides of the double-sided patch;
applying a power source onto the frame; attaching the power source to the main electrode and the counter electrode; and
attaching a conductive interfacing material onto each side of the main electrode and the counter electrode.
